(19) United States
(12) Patent Application Publication
(10) Pub. No.: US 2022/0062021 A1
(43) Pub. Date: Mar. 3, 2022

(54) Orthopedic Collar

(71) Applicant: Leon Rashed, San Pablo, CA (US)

(72) Inventor: Leon Rashed, San Pablo, CA (US)

(21) Appl. No.: 17/010,139

(22) Filed: Sep. 2, 2020

(51) Int. Cl. A61F 5/055

(52) U.S. Cl. CPC A61F 5/055

(57) ABSTRACT

An orthopedic collar includes a flexible tubular body having an upper edge, a lower edge, and a sidewall extending therebetween defining a central opening therethrough. A plurality of reinforcement members is disposed about an interior surface of the tubular body. A non-compressible member is affixed to a front side of the tubular body along an exterior surface thereof between the upper edge and the lower edge. The non-compressible member is disposed along a longitudinal axis of the tubular body and restricts vertical compression of the tubular body. In some embodiments, a flexible flange extends from the lower edge to support the tubular body on the shoulders of the user.

### BACKGROUND OF THE INVENTION

The present invention relates to orthopedic collars. More particularly, the present invention pertains to an orthopedic collar having a non-compressible member on a front side thereof, wherein a plurality of reinforcement members extends longitudinally along an interior surface of the collar.

Many individuals suffer from neck injuries which require the stabilization and support of the head and neck for treatment. Typically, such stabilization is provided by a neck brace worn about the neck that restricts the wearer's mobility, such that the wearer cannot easily tilt their head vertically or to one side. Frequently, these existing neck braces can be particularly bulky and uncomfortable, which can lead to difficulty sleeping or relaxing as the neck brace causes significant discomfort when worn. Often, typical neck braces include rigid plastic circumscribing the user's neck. Alternatively, resilient foam neck braces can be used in an effort to increase comfort at the cost of overall stability. While it is important to stabilize the head and neck of an individual with a neck injury, doing so while minimizing discomfort to the user is paramount, as treatments must typically be maintained over a long recovery period to be effective. Therefore, there is a need for a device that can provide a user with increased comfort while still restricting the vertical and lateral movement of the user's head.

In light of the devices disclosed in the known art, it is submitted that the present invention substantially diverges in design elements from the known art and consequently it is clear that there is a need in the art for an improvement to existing orthopedic collars. In this regard, the instant invention substantially fulfills these needs.

### SUMMARY OF THE INVENTION

In view of the foregoing disadvantages inherent in the known types of orthopedic collars now present in the known art, the present invention provides an orthopedic collar wherein the same can be utilized for providing comfort to the user while restricting neck movement in a manner consistent with treatment of head and neck injuries.

The present device comprises a flexible tubular body having an upper edge, a lower edge, and a sidewall extending therebetween defining a central opening therethrough. A plurality of reinforcement strips is disposed about an interior surface of the tubular body. A non-compressible member is affixed to a front side of the tubular body along an exterior surface thereof between the upper edge and the lower edge, wherein the non-compressible member is disposed along a longitudinal axis of the tubular body and is configured to restrict vertical compression of the tubular body. In this manner, a user wearing the orthopedic collar is prevented from significantly maneuvering their neck while providing a comfortable neck brace.

In some embodiments, a flexible flange extends from the lower edge and is configured to support the tubular body on the shoulders of the user. In some such embodiments, the flange is removably securable to the tubular body via complementary fastener disposed along each of an exterior of the tubular body at the lower edge thereof and an interior surface of the flange. In this way, the user can selectively decide whether the additional support of the flange is necessary. In another such embodiment, the flange is arcuate such that a linear distance between each of a front end of the flange and a rear end of the flange and the upper edge of the tubular body is greater than a linear distance between a central portion of the flange and the upper edge. In this manner, the flange better conforms to the shape of a user's shoulders to provide additional support to the tubular body.

In some embodiments, the sidewall comprises an arcuate form, such that a diameter of the tubular body at each of the upper edge and the lower edge is greater than at a midline thereof. In other embodiments, the tubular body is selectively moveable between an expanded position and a retracted position, wherein an inner diameter of the tubular body is greater when in the expanded position. In this way, the tubular body can expand to allow a user to insert their head through the central opening. In other embodiments, the tubular body is biased towards the retracted position, such that the tubular body secures comfortably about the user's neck.

In some embodiments, the plurality of reinforcement strips is disposed along a longitudinal axis of the tubular body, such that vertical compression of the tubular body is minimized. In another embodiment, the plurality of reinforcement strips is disposed at regular intervals about the interior surface, such that the entirety of the tubular body resists vertical compression consistently.

In another embodiment, the non-compressible member comprises a rigid structure, such that a user's ability to maneuver vertically along the front side of the tubular body is prevented. In other embodiments, a plurality of ridges is disposed along the non-compressible member between opposing lateral sides thereof, such that the non-compressible member is configured to flex slightly about the plurality of ridges. In this manner, the user is not able to substantially move their head vertically along the front side of the tubular body, however minimal common head movements are not uncomfortably restricted by the non-compressible member.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1A shows a perspective view of an embodiment of the orthopedic collar in the retracted position.

FIG. 1B shows a perspective view of an embodiment of the orthopedic collar in the expanded position.

FIG. 2 shows a cross-sectional view of an embodiment of the orthopedic collar.

FIG. 3 shows a close-up view of the non-compressible member of an embodiment of the orthopedic collar.

FIG. 4 shows an exploded view of an embodiment of the orthopedic collar with a flexible flange.

### DETAILED DESCRIPTION OF THE INVENTION

Reference is made herein to the attached drawings. Like reference numerals are used throughout the drawings to depict like or similar elements of the orthopedic collar. The figures are intended for representative purposes only and should not be considered to be limiting in any respect.

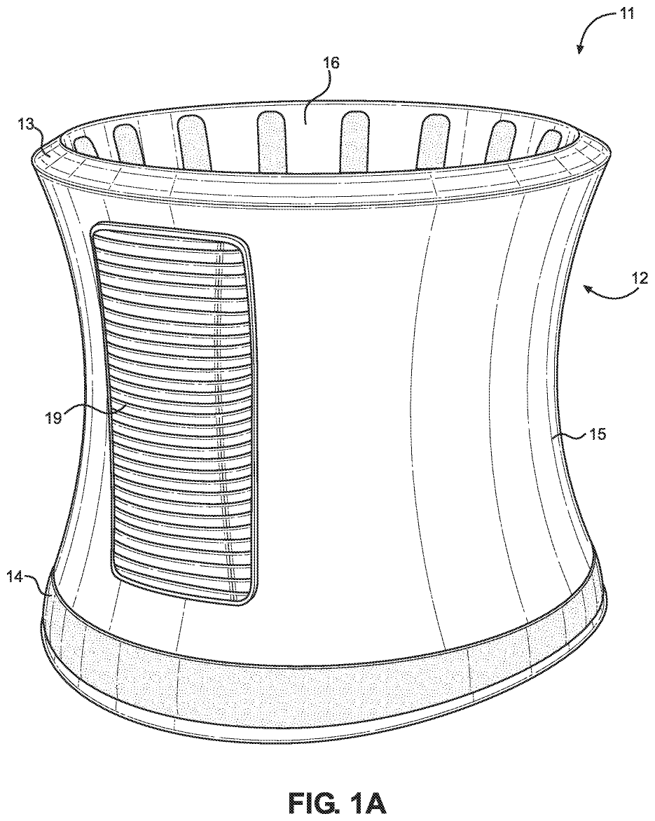

Referring now to FIGS. 1A and 1B, there is shown a perspective view of an embodiment of the orthopedic collar in the retracted position and a perspective view of an embodiment of the orthopedic collar in the expanded position, respectively. The orthopedic collar 11 comprises a tubular body 12 having an upper edge 13 disposed opposite a lower edge 14, wherein a sidewall 15 extends therebetween. A central opening 16 extends through the tubular body 12, wherein the central opening 16 is configured to receive a head of the user therethrough. A non-compressible member 19 is disposed on a front side of the tubular body 12, wherein the non-compressible member 19 is configured to further restrict a user's mobility along the front side of the tubular body 12. In the shown embodiment, the tubular body 12 comprises a flexible material configured to restrict a user's head movement, while allowing some compression of the sidewall 15 to provide increased comfort to the user. The tubular body 12 is contemplated to comprise a variety of flexible materials having properties commensurate with restricting mobility while retaining compressibility to provide comfort to the user, such as, but not limited to foam, silicon, reinforced fabric, and the like.

In the illustrated embodiments of FIGS. 1A and 1B, the tubular body 12 is configured to be selectively movable between an expanded position (as shown in FIG. 1B), and a retracted position (as shown in FIG. 1A). An inner diameter of the central opening 16 is configured to be greater when the tubular body 12 is in the expanded position than a diameter of the central opening 16 when the tubular body 12 is in the retracted position. In this manner, the user can move the tubular body 12 to the expanded position, such that a diameter of the central opening 16 is maximized, thereby allowing the orthopedic collar 11 to be placed over the user's head. In such embodiments, the diameter of the central opening 16 is configured to be less than a diameter of the user's head, such that the orthopedic collar 11 is secured about the user's neck and remains affixed to the user. In some embodiments, the tubular body 12 comprises an elastic material therein, such that the tubular body 12 is biased towards the retracted position. In this manner, once the tubular body 12 is expanded to be placed over the user's head, the tubular body 12 retracts automatically to restrict movement and provide support to the user's head.

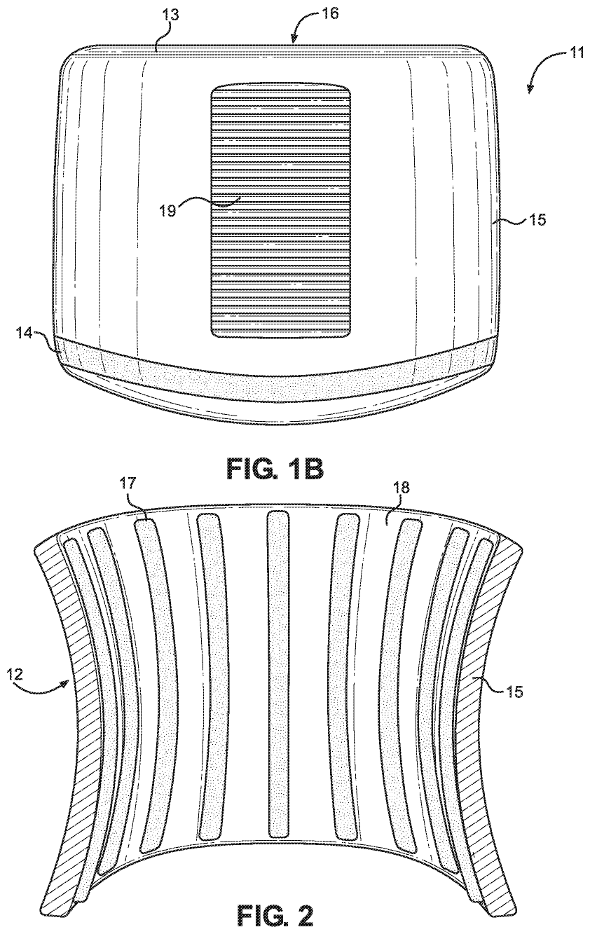

Referring now to FIG. 2, there is shown a cross-sectional view of an embodiment of the orthopedic collar. In the illustrated embodiment, the sidewall 15 comprises an arcuate shape, such that a diameter of each of the upper edge and the lower edge is greater than a diameter of the tubular body 12 at a midline thereof. In this manner, the flaring nature of the tubular body 12 provides increased support to the user as a larger surface area is in contact with the user along the upper and lower edges. Such an arrangement further provides additional comfort to the user as the forces placed on the tubular body 12 are evenly distributed along the greater surface area.

In the illustrated embodiment, a plurality of reinforcement strips 17 are disposed along an interior surface 18 of the tubular body 12. The plurality of reinforcement strips 17 are configured to provide increased structural integrity to the tubular body 12, such that the tubular body 12 retains a desired shape. In this manner, the plurality of reinforcement strips 17 serve to further restrict the user's range of motion as compression of the tubular body 12 is restricted thereby, further aiding the healing process of a neck injury. In the illustrated embodiment, the plurality of reinforcement strips 17 are disposed along a longitudinal axis of the tubular body 12. In the shown embodiment, the plurality of reinforcement strips 17 are disposed along the interior surface 18 at regular intervals, such that the plurality of reinforcement strips 17 are evenly distributed therealong. Such an arrangement ensures that the structural support added to the tubular body 12 by the plurality of reinforcement strips 17 is consistent across an entirety of the tubular body 12. In some embodiments, the plurality of reinforcement strips 17 are spring biased along the arcuate shape of the sidewall 15, such that the upper and lower edges of the tubular body 12 return to the retracted position after being placed in the expanded position.

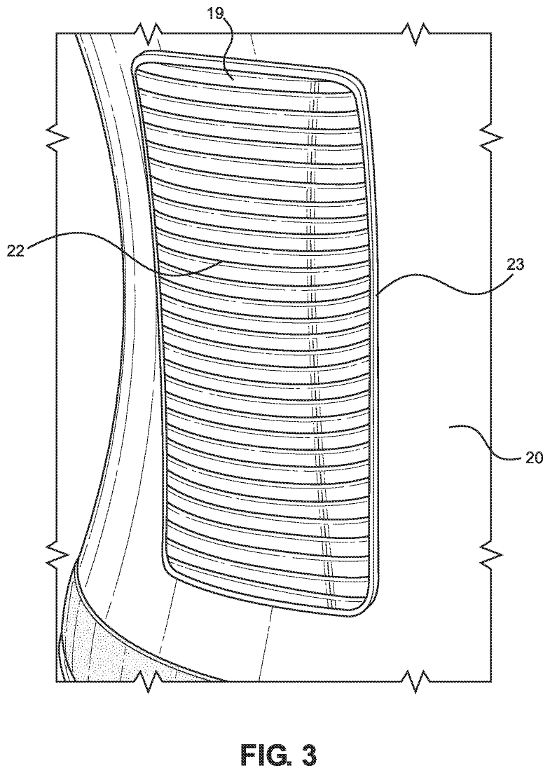

Referring now to FIG. 3, there is shown a close-up view of the non-compressible member of an embodiment of the orthopedic collar. The non-compressible member 19 is disposed on a front side 20 of the tubular body. The non-compressible member 19 is configured to restrict vertical movement of a user's head when the tubular body is worn, such that the user does not aggravate a neck injury. In the shown embodiment, the non-compressible member 19 is disposed between the upper and lower edges of the tubular body, such that a portion of the tubular body provides a cushion on opposing ends of the non-compressible member 19. In this manner, the user is provided maximal comfort, such that vertical movement of the head does not cause discomfort as the edges of the non-compressible member 19 contact the user. In some embodiments, the non-compressible member 19 is rigid, such that all vertical movement of the head is prevented, thereby ensuring strain on the user's injury is minimized. However, in the shown embodiment, the non-compressible member 19 further comprises a plurality of ridges 22 extending thereacross between opposing lateral sides 23 of the non-compressible member 19 such that a plurality of gaps is defined between the plurality of ridges 22. The plurality of ridges 22 are configured to allow the non-compressible member 19 to flex along the plurality of ridges 22, such that minor common head movements are not impeded thereby. In the shown embodiment, a depth of the non-compressible member 19 follows the contours of the arcuate sidewall, such that the depth is greatest at a central portion of the non-compressible member 19. As such, the depth of the non-compressible member 19 decreases towards each of an upper and lower end thereof. In this manner, the arcuate shape of the sidewall is bridged by the non-compressible member 19, thereby preventing the compression of the sidewall along the front side 20 thereof.

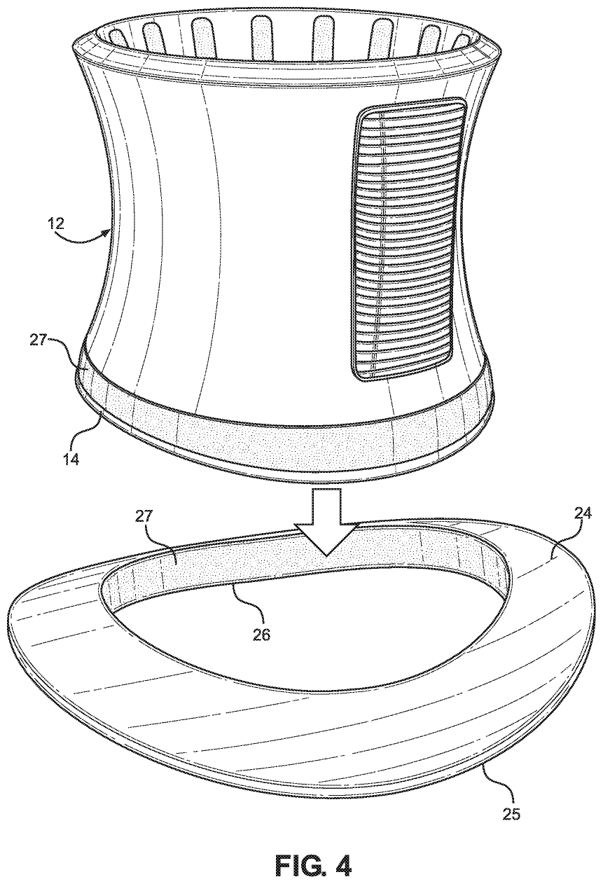

Referring now to FIG. 4, there is shown an exploded view of an embodiment of the orthopedic collar with a flexible flange. In the illustrated embodiment, the orthopedic collar further comprises a flange 24 configured to provide additional support and comfort to the user as the flange 24 braces the tubular body 12 against the shoulders of the user. In the shown embodiment, the flange 24 is removably securable to the lower edge 14 of the tubular body 12 via complementary fasteners 27 disposed along an outer surface of the lower edge 14 and an inner surface of the flange 24, respectively. In this manner, the user can selectively determine when the flange 24 is necessary for providing additional support, or whether the tubular body 12 is sufficient. In some embodiments, the flange 24 is permanently affixed to the tubular body 12 and extends from the lower edge 14 thereof. In the shown embodiment, the flange 24 is further arcuate, such that a linear distance between each of a front end 25 of the flange 24 and a rear end 26 of the flange 24 and the upper edge of the tubular body 12 is greater than that of a central portion of the flange 24. The arcuate nature of the flange 24 allows the flange 24 to better conform to the shape of a user, such that the front end 25 and rear end 26 are braced against a chest and back of the user, respectively.

In one use, the tubular body 12 is moved to the expanded position as shown in the illustrated embodiment of FIG. 1B. The tubular body 12 can then be lowered over the user's head and surround the user's neck to provide support and restrict movement thereof to promote healing of a neck injury. In embodiments having a flange 24, the flange 24 can be secured to the lower edge 14 of the tubular body 12, such that the tubular body 12 is further braced against the shoulders and torso of the user. The non-compressible member on the front side of the tubular body prevents the user from straining their neck via vertical head movements. The plurality of reinforcement strips further ensures that the tubular body 12 provides sufficient support to the head of the user while also restricting lateral and rearward head movements.

It is therefore submitted that the instant invention has been shown and described in various embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. An orthopedic collar, comprising:
a flexible tubular body having an upper edge, a lower edge, and a sidewall extending therebetween defining a central opening therethrough;
a plurality of reinforcement members disposed about an interior surface of the tubular body;
a non-compressible member affixed to a front side of the tubular body along an exterior surface thereof between the upper edge and the lower edge;
wherein the non-compressible member is disposed along a longitudinal axis of the tubular body and is configured to restrict vertical compression of the tubular body.

2. The orthopedic collar of claim 1, wherein the sidewall is arcuate such that a diameter of the tubular body at each of the upper edge and the lower edge is greater than at a midline thereof.

3. The orthopedic collar of claim 1, wherein the plurality of reinforcement strips is disposed along a longitudinal axis of the tubular body.

4. The orthopedic collar of claim 1, wherein the plurality of reinforcement strips is disposed at regular intervals along the interior surface.

5. The orthopedic collar of claim 1, wherein the tubular body is selectively movable between an expanded position and a retracted position, wherein an inner diameter of the tubular body is greater when in the expanded position.

6. The orthopedic collar of claim 5, wherein the tubular body is biased towards the retracted position.

7. The orthopedic collar of claim 1, wherein the non-compressible member comprises a rigid structure.

8. The orthopedic collar of claim 1, wherein the non-compressible member further comprises a plurality of ridges extending between opposing lateral sides thereof, such that the non-compressible member is configured to flex about the plurality of ridges.

9. An orthopedic collar, comprising:
a flexible tubular body having an upper edge, a lower edge, and a sidewall extending therebetween defining a central opening therethrough;
a plurality of reinforcement members disposed about an interior surface of the tubular body;
a non-compressible member affixed to a front side of the tubular body along an exterior surface thereof between the upper edge and the lower edge;
wherein the non-compressible member is disposed along a longitudinal axis of the tubular body and is configured to restrict vertical compression of the tubular body;
a flexible flange extending from the lower edge.

10. The orthopedic collar of claim 9, wherein the sidewall is arcuate such that a diameter of the tubular body at each of the upper edge and the lower edge is greater than at a midline thereof.

11. The orthopedic collar of claim 9, wherein the plurality of reinforcement strips is disposed along a longitudinal axis of the tubular body.

12. The orthopedic collar of claim 9, wherein the plurality of reinforcement strips is disposed at regular intervals along the interior surface.

13. The orthopedic collar of claim 9, wherein the tubular body is selectively movable between an expanded position and a retracted position, wherein an inner diameter of the tubular body is greater when in the expanded position.

14. The orthopedic collar of claim 13, wherein the tubular body is biased towards the retracted position.

15. The orthopedic collar of claim 9, wherein the non-compressible member comprises a rigid structure.

16. The orthopedic collar of claim 9, wherein the non-compressible member further comprises a plurality of ridges extending between opposing lateral sides thereof, such that the non-compressible member is configured to flex about the plurality of ridges.

17. The orthopedic collar of claim 9, wherein the flange is removably securable to the tubular body via complementary fasteners disposed along each of an exterior of the tubular body at the lower edge thereof and an interior surface of the flange.

18. The orthopedic collar of claim 9, wherein the flange is arcuate such that a linear distance between each of a front end of the flange and a rear end of the flange and the upper edge of the tubular body is greater than a linear distance between a central portion of the flange and the upper edge.
